(19) United States
(12) Patent Application Publication
(10) Pub. No.: US 2006/0015049 A1
(43) Pub. Date: Jan. 19, 2006

(54) Hip brace and abduction joint therefor

(76) Inventors: Daniel Suarez, Lawrenceville, GA (US); Brent Rosendahl, Tualatin, OR (US)

Correspondence Address: GARDNER GROFF, P.C., 2018 POWERS FERRY ROAD, SUITE 800, ATLANTA, GA, 30339, US

(21) Appl. No.: 10/891,170

(22) Filed: Jul. 14, 2004

(51) Int. Cl. A61F 5/00

(52) U.S. Cl. 602/23; 602/16; 602/24

(57) ABSTRACT

A hip brace apparatus for use by a user and comprising a waist cuff, a thigh cuff, and an abduction joint connecting the waist cuff to the thigh cuff, the abduction joint adapted for causing abduction of the user's leg as the user sits down while wearing the hip brace apparatus. The abduction joint comprises an upper pivot arm for attachment to the waist cuff and a lower pivot arm for attachment to the thigh cuff, with the lower pivot arm pivotally mounted for pivotal motion relative to the upper pivot arm about a pivot axis which extends at an oblique angle relative to the upper pivot arm. The pivot axis extends downwardly at an angle of between 100° and 115° relative to the upper pivot arm when the user is standing upright.

### FIELD OF THE INVENTION

The present invention relates generally to the field of orthopedic devices, and more particularly to a hip brace and abduction joint therefor.

### BACKGROUND OF THE INVENTION

Conventional orthopedic hip braces typically have a joint for connecting a waist cuff to a thigh cuff. It has been recognized that it is advantageous for the user's leg to be abducted. Abduction is the position the leg takes when it is held outwardly to the side. The advantage of this position is that it promotes keeping the ball of the thigh bone in the hip socket.

Conventional hip braces generally force the user's leg to be abducted at all times. In other words, conventional hip braces require the clinician to set the degree of abduction such that user's leg is constantly abducted, even while walking, which can be uncomfortable for the user and can cause the user to walk with an abnormal gait.

Therefore, it has been found that a need yet exists for an improved abduction joint for a hip brace that allows a user to ambulate normally but causes abduction of the user's leg as the user sits down while wearing the hip brace apparatus. It is to the provision of such an improved hip brace and abduction joint therefor meeting these and other needs that the present invention is primarily directed.

### SUMMARY OF THE INVENTION

Briefly described, in an illustrative form the present invention comprises a hip brace apparatus for use by a user and comprising a waist cuff, a thigh cuff, and an abduction joint connecting the waist cuff to the thigh cuff. The abduction joint is adapted for causing abduction of the user's leg as the user sits down while wearing the hip brace apparatus. The abduction joint can further comprise an upper pivot arm for attachment to the waist cuff and a lower pivot arm for attachment to the thigh cuff, with the lower pivot arm pivotally mounted for pivotal motion relative to the upper pivot arm about a pivot axis which extends at an oblique angle relative to the upper pivot arm. Preferably, the pivot axis extends at an angle of between 90° and 120° relative to the upper pivot arm, and more preferably, the pivot axis extends at an angle of about 110° relative to the upper pivot arm. Also preferably, the pivot axis extends in a generally downwardly direction when the user (wearer) is standing upright.

Moreover, the abduction joint can comprise a pivot axle extending from the upper pivot arm and a pivot plate coupled to the lower pivot arm or formed with the lower pivot arm, with the pivot axle extending through the pivot plate. The hip brace apparatus can also include adjustable pivot limit stops for limiting the maximum pivotal motion of the thigh cuff in both the forward and backward directions. The adjustable limit stops can be repositioned in different positions on the pivot plate. Additionally, the lower pivot arm comprises a lateral adjustable pivot for adjusting the lateral orientation of the thigh cuff.

In another aspect, the present invention comprises a hip brace apparatus for use by a user and includes a waist cuff, a leg cuff, an upper member for attachment to the waist cuff, and a lower member for attachment to the leg cuff. The lower member is pivotally mounted to the upper member for pivotal movement with respect thereto about a pivot axis extending at an oblique angle relative to the upper member. Thus, as the user sits down while wearing the hip brace apparatus, the user's leg is abducted. Preferably, the oblique angle is between 100° and 115°.

In yet another aspect, the present invention comprises an improvement for a hip brace apparatus of the type for use by user and having a body cuff, a leg cuff, and a joint connecting the body cuff with the leg cuff to allow movement of the leg cuff. The improvement comprises the joint being adapted to cause substantial abduction of the leg as the user sits down while wearing the hip brace apparatus. The joint further includes an upper member and a lower member pivotally mounted to the upper member for pivotal movement about a pivot axis, with the pivot axis being oriented at an oblique angle relative to the upper member.

In still another aspect, the present invention comprises an abduction joint comprising a first attachment bracket defining a longitudinal axis extending therethrough and a pivot axle coupled to the first attachment bracket, wherein the pivot axle defines a pivot axis extending at an oblique angle relative to the longitudinal axis.

These and other objects, features, and advantages of the present invention will become more apparent upon reading the following specification in conjunction with the accompanying drawing figures.

### BRIEF DESCRIPTION OF THE DRAWING FIGURES

FIG. 1 is a perspective view of a hip brace apparatus according to a preferred embodiment of the present invention.

FIG. 2 is a perspective view of an abduction joint portion of the apparatus of FIG. 1 in accordance with a preferred embodiment of the present invention.

FIG. 3 is a perspective, exploded view of the abduction joint of FIG. 2.

FIG. 4 is a side, exploded view of the abduction joint of FIG. 2.

FIG. 5 is a side view of the abduction joint of FIG. 2.

FIG. 6 is a detailed, side view of a portion of the abduction joint of FIG. 5.

FIG. 7 is a detailed, perspective view of a boss portion of the abduction joint of FIG. 2 having a pivot axle thereon.

FIG. 8A depicts a schematic view of the hip brace of FIG. 1 when the user is standing upright, and FIG. 8B depicts a schematic view of the hip brace of FIG. 1 showing abduction of the user's leg when the user sits down.

### DETAILED DESCRIPTION OF THE PREFERRED EMBODIMENTS

Referring now to the drawing figures, in which like reference numbers refer to like parts throughout the several views, preferred forms of the present invention will now be described by way of example embodiments. It is to be understood that the embodiments described and depicted herein are only selected examples of the many and various forms that the present invention may take, and that these examples are not intended to be exhaustive or limiting of the claimed invention. Also, as used in the specification including the appended claims, the singular forms “a,” “an,” and “the” include the plural unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

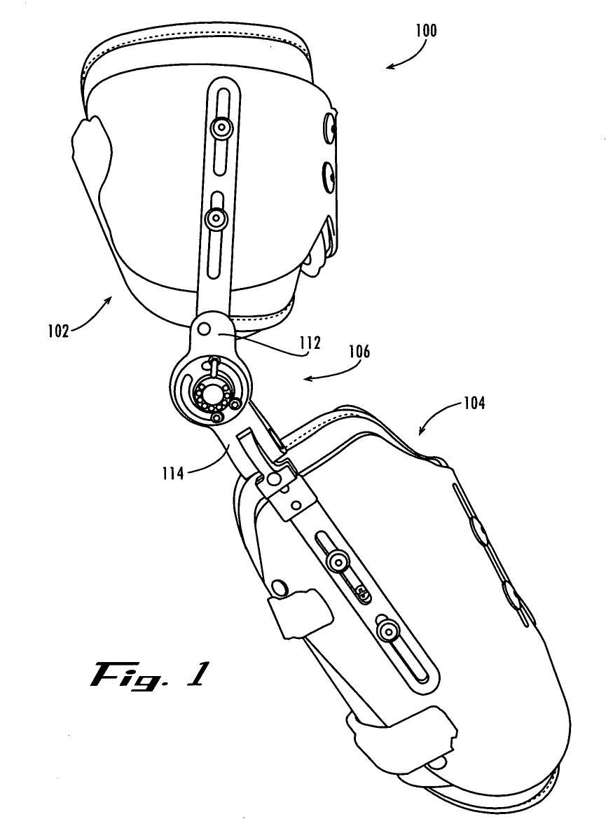

As shown in FIG. 1, an orthopedic hip brace 100 includes a body or waist cuff 102 and a leg or thigh cuff 104 connected together by a joint indicated generally at 106. Preferably, the waist cuff 102 is sized to fit snugly and securely around the waist or lower abdomen of the user of the brace 100. Similarly, the thigh cuff 104 is sized to fit snugly and securely around the user's thigh. It should be noted that the thigh cuff 104 can be adapted to fit either the user's right or left thigh.

Preferably, the waist cuff 102 and the thigh cuff 104 each preferably have a rigid portion constructed of a durable material such as a molded plastic including high density polyethylene (HDPE), metallocene polyethylene (mPE), or low density polyethylene (LDPE). It should be understood that other durable and rigid materials can be employed without departing from the scope of the present invention. The first portions of the waist cuff 102 and the thigh cuff 104 can be modular such that they can be adapted to fit different body shapes. Additionally or alternatively, a differently sized hip brace can be made to accommodate persons of different builds, such as for those having small, medium, large, or extra-large builds.

Optionally, the waist cuff 102 and the thigh cuff 104 can include soft liner pads. Preferably, the liner pads are breathable and are capable of absorbing perspiration and reducing heat buildup. Also preferably, the liner pads are removably secured to the waist cuff 102 and the thigh cuff 104 with for example, but not limited to, hook and loop material, snaps, buttons, or adhesive.

Preferably, the waist cuff 102 and the thigh cuff 104 have a strap or plurality of straps to secure the waist cuff and thigh cuff to the user's body. The straps can be secured with hook and loop material, buckles, buttons, zippers, or snaps, to name a few.

FIGS. 2-6 show details of one form of the abduction joint 106 for connecting the waist cuff 102 to the thigh cuff 104. Preferably, the joint 106 is positioned between two struts or attachment brackets, an upper strut 112 and a lower strut 113. The upper strut 112 can be attached to an outer face of the waist cuff 102. The upper strut 112 also defines a longitudinal axis 118 extending therethrough (see FIG. 3). The lower strut 113 can be attached to an outer face of the thigh cuff 104.

In the preferred embodiment, the struts 112 and 113 are shanks, each having a pair of elongated fastener slots 114, 115 and 116, 117 therein. The elongated slots 114 and 115 are used to attach the upper strut 112 of the joint 106 securely to the waist cuff 102 by receiving fasteners therein. The elongated slots 116 and 117 are used to attach the lower strut 113 securely to the thigh cuff 104 by receiving fasteners therein. The elongated slots 114 and 115 also allow the height of the joint 106 to be adjusted relative to the waist cuff 102. Similarly, the elongated slots 116 and 117 allow the height of the joint 106 to be adjusted relative to the thigh cuff 104. Although the struts 112, 113 have been described and depicted herein in terms of shanks, it should be understood by those skilled in the art that other shapes and sizes can be employed without departing from the scope of the present invention.

In the preferred embodiment, the pair of struts 112 and 113 can also be considered pivot arms in which the lower pivot arm 113 is pivotally mounted for pivotal movement relative to the upper pivot arm 112 about a pivot axis 120 (see FIG. 3), which extends at an oblique angle α relative to the upper pivot arm 112 and laterally relative to the waist cuff 102 (neither fore nor aft). Preferably, the angle α is an obtuse angle of between about 90° to about 120° relative to the upper pivot arm 112. More preferably, the angle α is between about 100° to about 115° relative to the upper pivot arm 112. Even more preferably, the angle α is about 110° relative to the upper pivot arm 112.

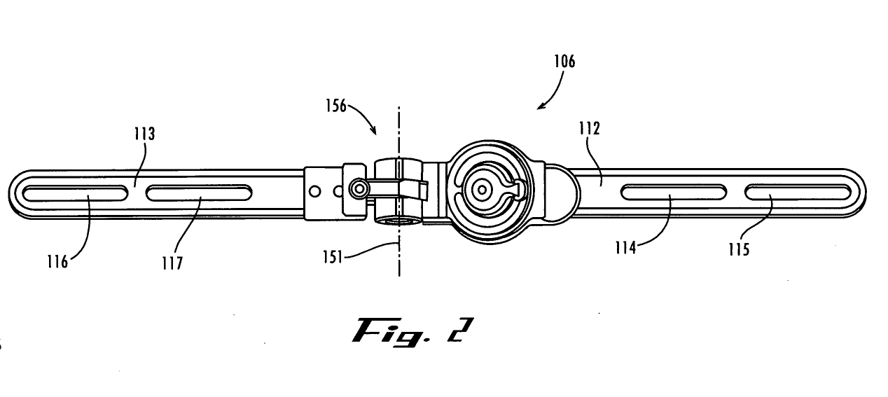

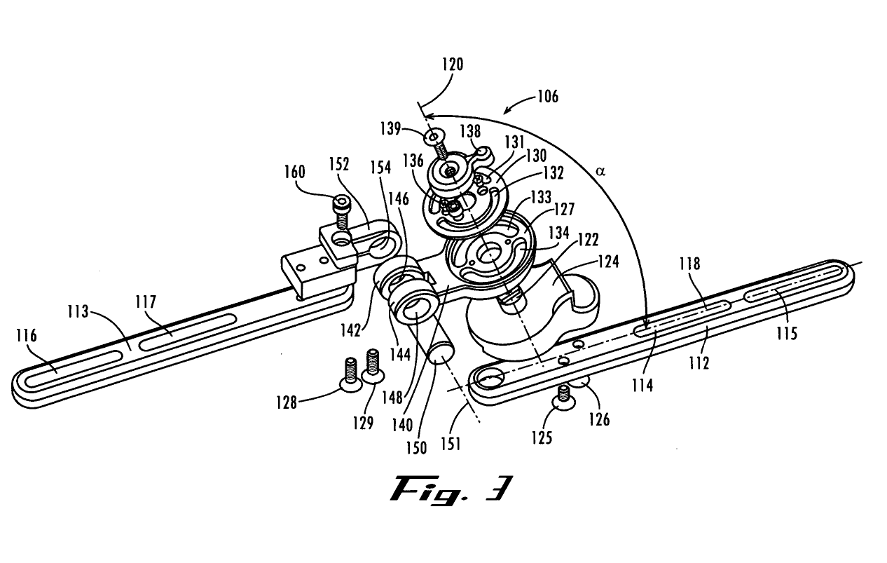

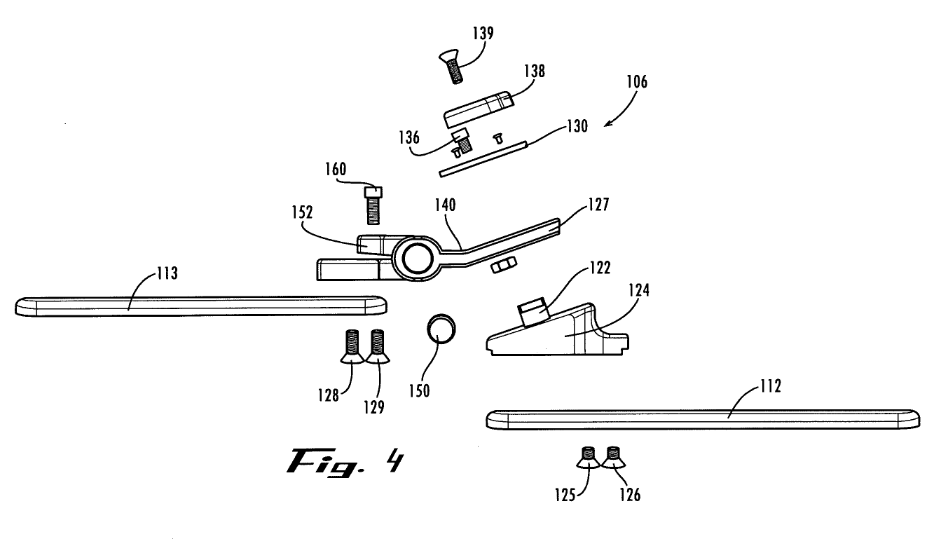

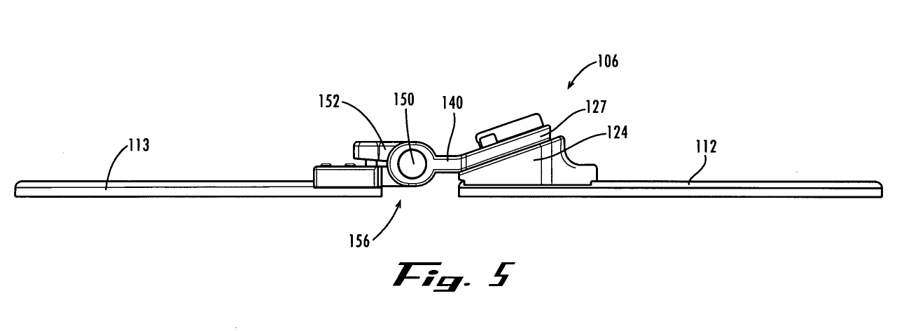

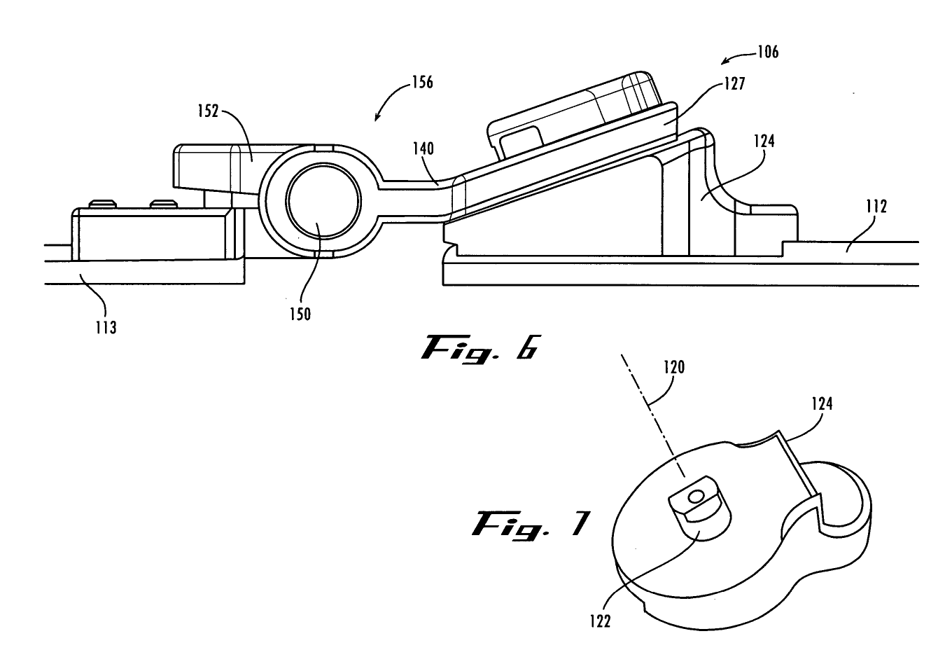

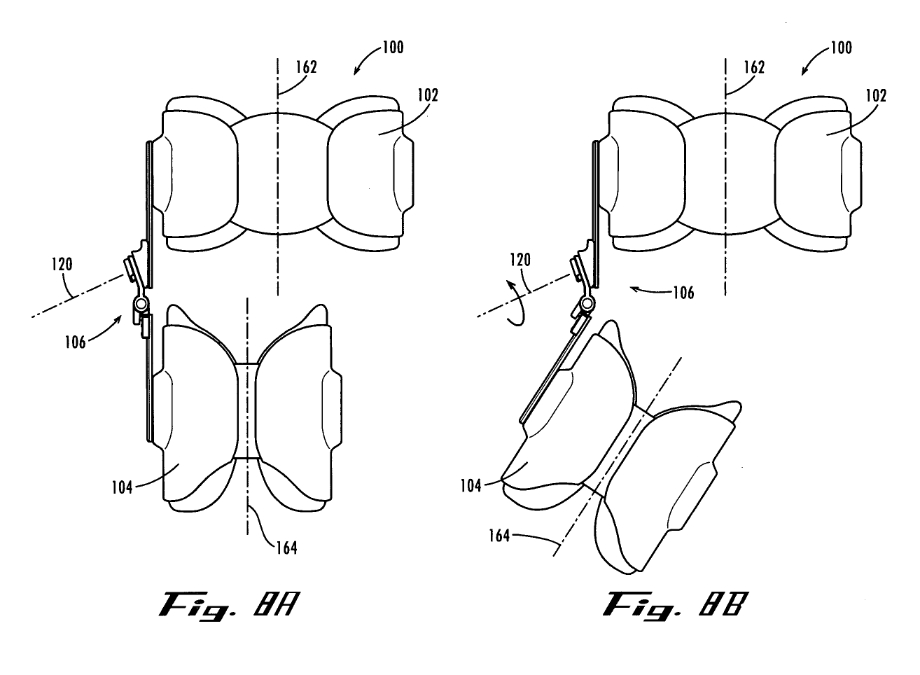

As seen in FIGS. 3 and 7, the joint 106 also includes a pivot axle 122 extending from a boss 124 and along the pivot axis 120. The boss 124 can be coupled to or formed as a part of the upper strut 112. In one form, the boss 124 can be coupled to the strut 112 with a pair of screws 125 and 126 or fasteners. Alternately, the upper strut 112 and the boss 124 having the pivot axle 122 thereon can be an integral element. The joint 106 also includes a pivot plate 127 coupled to or formed as a part of the lower strut 113. In one form, the pivot plate 127 can be secured to the lower strut 113 with screws 128 and 129 or fasteners. Alternatively, the pivot plate 127 and the lower strut 113 can be an integral element.

Preferably, the joint 106 further comprises a second plate 130 having a pair of arcuate slots 131, 132 therein that align with a pair of arcuate slots 133, 134 in the pivot plate 127. Within each aligned pair of arcuate slots is a limit stop screw 136 (along with an unshown nut) which is movable along the arc of the slot and securable therein. That is, the limit stop screws 136 can be repositioned in different positions on the pivot plate 127 and then fixed in place. Secured to the second plate 130 is a limit finger on cog 138, which can engage the limit stop screws 136 so as to limit maximum pivotal movement about the pivot axis 120. The cog 138 is secured to the pivot axle with a screw 139 such that the plate 127 can move relative to both the pivot axle 122 and the cog 138. Thus, the limit stop screws 136 and the cog 138 together limit the maximum pivotal movement of the thigh cuff 104 in both the forward and backward directions.

In the preferred embodiment, the pivot plate 127 is part of a knuckle 140. Preferably, the knuckle 140 has two collars 142 and 144 at an end thereof, with a gap therebetween. The collars 142 and 144 have co-aligned bores 146 and 148 extending therethrough for receiving a pin 150 therein for an interference fit. An adjustable crook bracket 152 having a bore 154 therethrough is interposed between the two collars 142 and 144. The bores 146 and 148 of the collars 142 and 144 are aligned with the bore 154 of the crook bracket 152, and a pin 150 is driven therethrough so as to create an interference fit between the pin and the collars. Optionally, the pin 150 can be knurled.

Collectively, the knuckle 140 and the adjustable crook bracket 152 form a lateral adjustable pivot 156 for adjusting the lateral orientation of the thigh cuff 104 (see FIGS. 2 and 5). The adjustable pivot 156 allows the lower strut 113 to be pivoted about a pivot axis 151, as seen in FIGS. 2 and 3. The crook bracket 152 has an adjustable screw 160 to lock the pivot 156 at a desired angle. To adjust the pivot angle, the screw 160 can be loosened, and then the angle can be adjusted prior to locking the pivot 156 by tightening the screw 160.

As seen in FIGS. 8A and 8B, when the user is wearing the hip brace 100 and is standing upright, a torso elongation axis 162 is generally parallel to a leg elongation axis 164. The pivot axis 120 of the joint 106, and hence the pivot axle 122, point in a generally downward direction. Thus, as the user begins to sit down, the user's leg begins to bend toward a 90° angle while the joint 106 pivots about the pivot axis 120 in the direction of the arrow. Thus, the joint 106 causes the user's leg to abduct outwardly as seen in FIG. 8B, which in turn promotes keeping the ball of thigh bone in its hip socket.

While the invention has been described with reference to preferred and example embodiments, it will be understood by those skilled in the art that a variety of modifications, additions and deletions are within the scope of the invention, as defined by the following claims.

What is claimed is:

1. A hip brace apparatus for use by a user and comprising:
a waist cuff;
a thigh cuff; and
an abduction joint connecting the waist cuff to the thigh cuff, the abduction joint adapted for causing abduction of the user's leg as the user sits down while wearing the hip brace apparatus.

2. A hip brace apparatus as claimed in claim 1 wherein the abduction joint comprises an upper pivot arm for attachment to the waist cuff and a lower pivot arm for attachment to the thigh cuff, with the lower pivot arm pivotally mounted for pivotal motion relative to the upper pivot arm about a pivot axis which extends at an oblique angle relative to the upper pivot arm.

3. A hip brace apparatus as claimed in claim 2 wherein the pivot axis extends at an angle of between 90° and 120° relative to the upper pivot arm.

4. A hip brace apparatus as claimed in claim 2 wherein the pivot axis extends at an angle of between 100° and 115° relative to the upper pivot arm.

5. A hip brace apparatus as claimed in claim 2 wherein the pivot axis extends at an angle of about 110° relative to the upper pivot arm.

6. A hip brace apparatus as claimed in claim 2 wherein the abduction joint comprises a pivot axle extending from the upper pivot arm and a pivot plate coupled to or formed with the lower pivot arm, with the pivot axle extending through the pivot plate.

7. A hip brace apparatus as claimed in claim 6 further comprising adjustable pivot limit stops for limiting the maximum pivotal motion of the thigh cuff in forward and backward directions.

8. A hip brace apparatus as claimed in claim 7 wherein the adjustable pivot limit stops comprise movable fasteners which can be repositioned in different positions on the pivot plate.

9. A hip brace apparatus as claimed in claim 2 wherein the lower pivot arm comprises a lateral adjustable pivot for adjusting the lateral orientation of the thigh cuff.

10. A hip brace apparatus as claimed in claim 2 wherein the pivot axis extends laterally relative to the waist cuff.

11. A hip brace apparatus for use by a user and comprising:
a waist cuff;
a leg cuff;
an upper member for attachment to the waist cuff; and
a lower member for attachment to the leg cuff, the lower member being pivotally mounted to the upper member for pivotal movement with respect thereto about a pivot axis extending at an oblique angle relative to the upper member.

12. A hip brace apparatus as claimed in claim 11 wherein as the user sits down while wearing the hip brace apparatus, the pivot axis being at an oblique angle causes the user's leg to be abducted.

13. A hip brace apparatus as claimed in claim 11 wherein the oblique angle is between 100° and 115° relative to the upper member.

14. A hip brace apparatus for use by a user and comprising:
a body cuff;
a leg cuff; and
abduction joint means pivotally coupling the body cuff to the leg cuff for causing abduction as the user sits down while wearing the hip brace apparatus.

15. The hip brace apparatus of claim 14, wherein the abduction joint means comprises a first strut for attaching to the body cuff and having a pivot axle coupled thereto.

16. The hip brace apparatus of claim 15, wherein the first strut defines a longitudinal axis extending therethrough and the pivot axle defines a pivot axis extending at an oblique angle relative to the longitudinal axis.

17. The hip brace apparatus of claim 15, wherein the first strut defines a longitudinal axis extending therethrough and the pivot axle defines a pivot axis extending at an obtuse angle relative to the longitudinal axis.

18. In a hip brace apparatus of the type for use by user and having a body cuff, a leg cuff, and a joint connecting the body cuff with the leg cuff to allow movement of the leg cuff, the improvement therein comprising the joint being adapted to cause substantial abduction of the leg as the user sits down while wearing the hip brace apparatus.

19. The improvement of claim 18 wherein the joint includes an upper member and a lower member pivotally mounted to the upper member for pivotal movement about a pivot axis, with the pivot axis being oriented at an oblique angle relative to the upper member.

20. The improvement of claim 19, wherein the oblique angle is an obtuse angle relative to the upper member.

21. The improvement of claim 19, wherein the oblique angle is between 90° and 120° relative to the upper member.

22. The improvement of claim 19, wherein the oblique angle is between 100° and 115° relative to the upper member.

23. The improvement of claim 19, wherein the oblique angle is about 110° relative to the upper member.

24. An abduction joint, comprising:
a first attachment bracket defining a longitudinal axis extending therethrough; and
a pivot axle coupled to the first attachment bracket, wherein the pivot axle defines a pivot axis extending at an oblique angle relative to the longitudinal axis.

25. The abduction joint of claim 24, wherein the oblique angle defined between the pivot axis and the longitudinal axis is between 90° and 120°.

26. The abduction joint of claim 24, wherein the oblique angle defined between the pivot axis and the longitudinal axis is between 100° and 115°.

27. The abduction joint of claim 24, wherein the oblique angle defined between the pivot axis and the longitudinal axis is about 110°.

28. The abduction joint of claim 27, and further comprising a second attachment bracket coupled to or formed with a pivot plate for receiving a pivot axle extending from the first attachment bracket, wherein the pivot axle defines the pivot axis extending at an oblique angle relative to the longitudinal axis.

29. An abduction joint, comprising:
a first flange; and
a second flange pivotally mounted for pivotal motion relative to the first flange about a pivot axis which extends at an oblique angle relative to the first flange.

30. A hip brace apparatus for use by a user and comprising:
a body cuff;
a leg cuff; and
an abduction joint connecting the waist cuff to the thigh cuff, the abduction joint adapted for causing abduction of the user's leg as the user sits down while wearing the hip brace apparatus,
wherein the abduction joint comprises a pivot axle extending from the upper pivot arm, with the pivot axle extending generally downwardly when the user is in a standing position.
